(19) United States
(12) Patent Application Publication
(10) Pub. No.: US 2018/0105570 A1
(43) Pub. Date: Apr. 19, 2018

(54) METHOD FOR PREPARATION OF A RECOMBINANT PROTEIN FROM A PRECUSOR

(71) Applicant: MABION SA, Kutno (PL)

(72) Inventors: Julita Balcerek, Reczno (PL); Katarzyna Sznilik, Czestochowa (PL); Slawomir Jaros, Lodz (PL); Maciej Wieczorek, Kielpin (PL)

(21) Appl. No.: 15/532,811

(22) PCT Filed: Dec. 4, 2015
(86) PCT No.: PCT/PL2015/000197

§ 371 Date: Jun. 2, 2017

(30) Foreign Application Priority Data
Dec. 5, 2014 (PL) P.410410

(51) Int. Cl. C07K 14/62; C12P 21/06

(52) U.S. Cl. CPC C07K 14/62; C12Y 304/21061; C12P 21/06; C12Y 304/21004; C12Y 304/21009; C12Y 304/17002

(57) ABSTRACT

The method for preparation of a recombinant protein from a precursor, preferably mammalian insulins, including human insulins and their analogues, characterised in that a protease is used which hydrolyses one or more peptide bonds in this protein, wherein the protease disrupts the peptide bond from the C-end side of a basic amino acid, when the amino acid is the second one after other basic or neutral amino acid, and such an order enables specific recognition of both amino acids by the protease. The invention is applicable in biotechnology and pharmaceutical industry.

### BACKGROUND

More than 382 million people suffer from diabetes worldwide, among them 56 million are citizens of Europe. According to the prognoses made by international statistical offices, in 2035 the incidence will increase to about 592 million. Most people affected with diabetes are between 40-59 years old and in 80% they live in low- and medium-developed countries. In 2013, there were 5.1 million deaths reported worldwide due to this disease, which is an 11% increase compared to the data from 2011. For example, according to the most recent statistics from the National Vital Statistics Reports, in 2010 in the United States alone, 69 thousand people died because of it. Diabetes is a disease caused by the impaired pancreatic activity resulting in lower production of the hormone called insulin which regulates the blood sugar level (diabetes type I, called insulin-dependent) or in the lack of the organism's ability to use produced insulin (diabetes type II). According to WHO, in 2030 diabetes will be the seventh cause of death worldwide. The above epidemiological and technical data unambiguously show how important is the progress in therapeutic recombinant proteins development without which therapies of diseases such as diabetes would be less effective.

In order to understand the advantages of exemplary embodiments, it is important to go through insulins and their analogues production methods available thus far. The traditional technology of insulin production was based on the extraction of this hormone from animal tissues and subsequent chromatographic purification. To inactivate and remove trypsin, an enzyme which could degrade insulin, bovine or porcine pancreases were cut, treated with ethanol and acidified to pH of 2 using HCl or H₂SO₄. Then, calcium carbonate was added for neutralisation, followed by condensation of the solution under vacuum in low temperatures, and then precipitation with salt, dissolution in water and reprecipitation by adjusting pH to insulin isoelectric point.

The need for the production of typically human insulin resulted in the development of various technologies for obtaining it. The first approach was the hormone extraction from human pancreas, though in practice it is possible only on an analytical scale. Other proposal was the chemical synthesis whose disadvantages definitely are the necessity to bind two separately synthesized insulin chains and high costs. Eventually, two following approaches have proven to be useful, namely converting the porcine insulin to a human one and obtaining recombinant insulin. The technology of human insulin production based on the conversion, on a commercial scale, was developed by Novo Nordisk and included substituting B-30 position alanine in porcine insulin by threonine in five steps. Firstly, insulin is extracted from frozen porcine pancreases, then purified porcine insulin is converted to a human one in a medium which comprises small quantities of water and trypsin and large quantities of organic solvents and threonine esters. Trypsin hydrolyses insulin between Lys29-AlaB30 positions, simultaneously it catalyses the reaction in which threonine ester substitutes alanine at B-30 position. After the enzymatic reaction, the chromatographic purification is conducted in order to remove proinsulin and other reagents, and then formulating and portioning the product out is performed in sterile conditions. Currently, however, the most common method for obtaining insulins is the employment of recombinant DNA technology. Companies Eli Lilly and Co. (Indianapolis) and Genentech (San Francisco) were pioneers in this field and together they developed the first recombinant insulin. In 1978, when the hormone expression was obtained in Escherichia coli K12 cells using the pBR322 expression vector, the scale of the process was successfully increased, and in 1982 the marketing authorization was granted. The first process was based on obtaining A and B chain as fusion proteins with β-galactosidase in separate bacterial cultures. These products were intracellular, they were present in cytoplasm as inclusion bodies. After the recovery from inclusion bodies, fusion protein molecules were subjected to digestion with CNBr which occurred with methionine separating β-galactosidase from insulin chain, and then were purified. The next step was mixing A and B chain in a 2:1 ratio (S-sulfonated forms) in the presence of mercaptan. After 24 hours, the efficiency of insulin synthesis was around 60% in reference to the quantity of B chain. A significant disadvantage of this technology was the limited efficiency in obtaining A and B chains only (with the length of 21 or 30 amino acids, respectively) caused by a huge mass disproportion regarding β-galactosidase attached to them (around 1000 amino acids). Further development of the insulin production technology was directed to increase the efficiency of the A and B chain production through the shortening of fusion peptides, e.g. changing lac operon (β-galactosidase system) into a tryptophan one (Trp) in which the fusion protein had only around 190 amino acids.

The newest technological approach is the usage of recombinant proinsulin built out of A, B and C peptides from one cell clone, and then enzymatic treatment resulting in the conversion of proinsulin to insulin. It has many advantages, one fermentation process and then one purification process of the protein after fermentation is enough, instead of two separate ones for A and B chains. This approach was used for the first time in the industrial scale production in 1986.

Enzymatic treatment most frequently consists in using two enzymes, trypsin and carboxypeptidase B. Trypsin is a serine protease which cuts peptide bonds from the C-end side of positively charged amino acids (arginine and lysine), if the next amino acid is not proline. In case of human proinsulin, trypsin disrupts a peptide bond between lysine and arginine on the C-end of recombinant proinsulin, carboxypeptidase B cuts off basic amino acids on the C-end which resulted from trypsin activity. The disadvantage of this approach is the fact that the cutting will also result in various insulin derivatives being contaminants, such as A21-desamido insulin, des-threonine-(B30) insulin, arginyl-(A0) insulin i diarginyl-(B31,B32) insulin.

The most significant ones among these contaminants are desamido insulin and des-threonine insulin (result of Thr(B30) elimination by trypsin). A large problem is that des-threonine insulin has the same net charge as insulin. The only difference are small differences in hydrophobicity, thus removing this contaminant is possible in a few chromatographic steps. One possibility to minimize contaminants of such a type is to block the formation of des-threonine insulin by citraconilation, but this would be an additional step in the process.

Recently, insulin analogues which give various additional clinical benefit in comparison to normal human insulin have become more and more clinically significant. The most important ones from the market point of view are Humalog—name of the active substance being insulin lispro (Eli Lilly), which appeared on the market in 1996, NovoMix—with the active substance of insulin aspart (NovoNordisk, 1999), and Lantus—with the active substance of insulin glargine (Sanofi Aventis, 2000). Insulin lispro is an analogue with accelerated activity characterised by the changed order of amino acids in positions B28 (proline) and B29 (lysine). Aspart is characterised by B28 proline substituted with aspartic acid and is also an analogue with accelerated activity. Insulin glargine has A21 asparagine changed into glycine and B chain elongated with two arginine. Its significant advantage is the prolonged activity which translates to the increase in patient's comfort thanks to the reduction in injection number to one per day. Insulin analogues are obtained by using recombinant DNA technology. Changes in the amino acid order or substitutions are not a huge problem, simple sequence engineering is enough, the remaining part of the process can stay unchanged. It is much more difficult to obtain analogues with amino acids added at ends, and particularly basic amino acids, e.g. insulin glargine. In that case, it is necessary to use trypsin and then additional chemical modifications and to attach arginine.

P 391 975 application discloses the use of other enzymes than trypsin and carboxypeptidase B for selective digestion of proinsulin and precursors of human insulin analogues. The disclosed process for obtaining proteins from precursors thereof, including insulin and its analogues, using enzymes other than trypsin and carboxypeptidase B consisted in introducing to the precursor one or more amino acids recognised by proteases/peptidases, and then using these enzymes for removing redundant fragments and forming the actual target protein. The use of enterokinase and Asp-N endoproteinase in case of proinsulin or proinsulin analogue conversion to insulin or insulin analogue, respectively, allows to obtain a molecule of insulin or its analogue. Enterokinase with high specificity recognises amino acid sequence (DDDDK) which was introduced for it between the C′-end of C-peptide and A chain. With the help of Asp-N endoproteinase and by introducing the additional amino acid (D=Asp), recognised by protease, insulin B chain was separated from the N′-end of C-peptide. Hydrolysis of the peptide bond at this site occurs specifically.

### SUMMARY

Exemplary methods for preparation of a recombinant protein from a precursor, such as mammalian insulins, including human insulins and their analogues, by highly specific proteolysis in extracellular conditions (in vitro) using serine protease other than trypsin or serine protease other than trypsin and carboxypeptidase B.

### BRIEF DESCRIPTION OF THE DRAWINGS

FIG. 1 shows a scheme of human proinsulin precursor structure.

FIG. 2 shows a scheme of insulin precursor proteolysis with trypsin and carboxypeptidase B.

FIG. 3 shows a chromatogram of the separation of P_mMabionlGlargine_1 proteolysis products, C4 (HPLC).

FIG. 4 shows a chromatogram of the separation of P_mMabionlGlargine_1 proteolysis products (MS).

FIG. 5 shows the efficiency of P_mMabionlGlargine_1 proteolysis with Kex2 protease-proteolysis over time.

FIG. 6 shows a scheme of insulin glargine precursor proteolysis with Kex2

FIG. 7 shows a scheme of insulin analogue (Lispro) precursor proteolysis with Kex2 and carboxypeptidase

FIG. 8 shows results from the analysis of P_mMabionLGlargine_1 insulin secondary structure in comparison with insulin glargine (Sanofi Aventis).

FIG. 9 shows insulin glargine after proteolysis and purification reaction with the use of a single chromatographic step.

FIG. 10 shows the assessment of proteolysis products mass and the comparison of obtained values with theoretical ones.

FIG. 11 shows a scheme of insulin proteolysis with trypsin and carboxypeptidase B (citraconylation).

FIG. 12 shows a scheme of insulin precursor proteolysis with enterokinase and endopeptidase Asp-N.

FIG. 13 shows a scheme of Met-His-Proins structure.

FIG. 14 shows a visualisation of the separation of the products from P_mMabionlGlargine_1 enzymatic proteolysis in 18% tricine gel.

FIG. 15 shows a chromatogram of the separation of the products from Met-His_Proins proteolysis, C4 (MS).

FIG. 16 shows the stability and specificity of P_mMabionlGlargine_1 proteolysis over longer time than optimal.

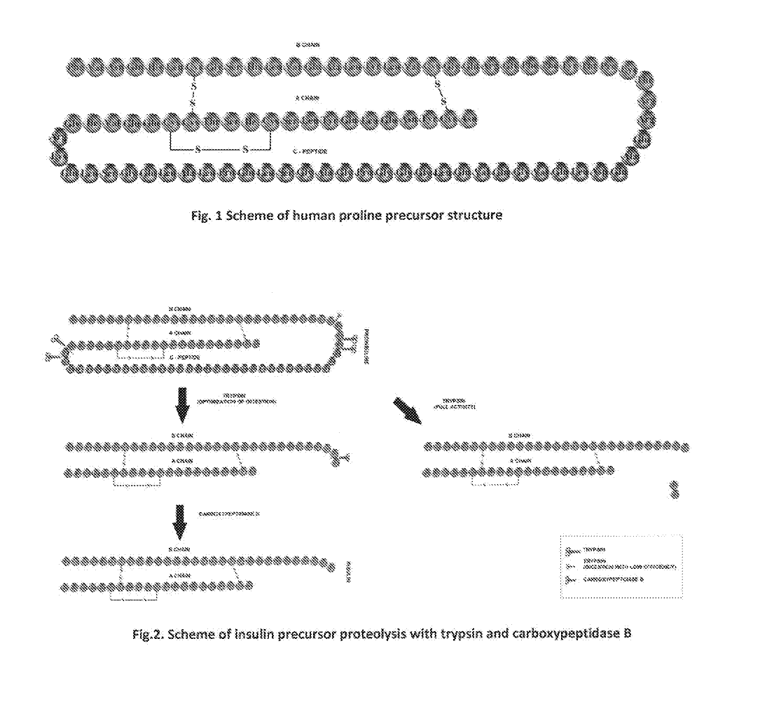

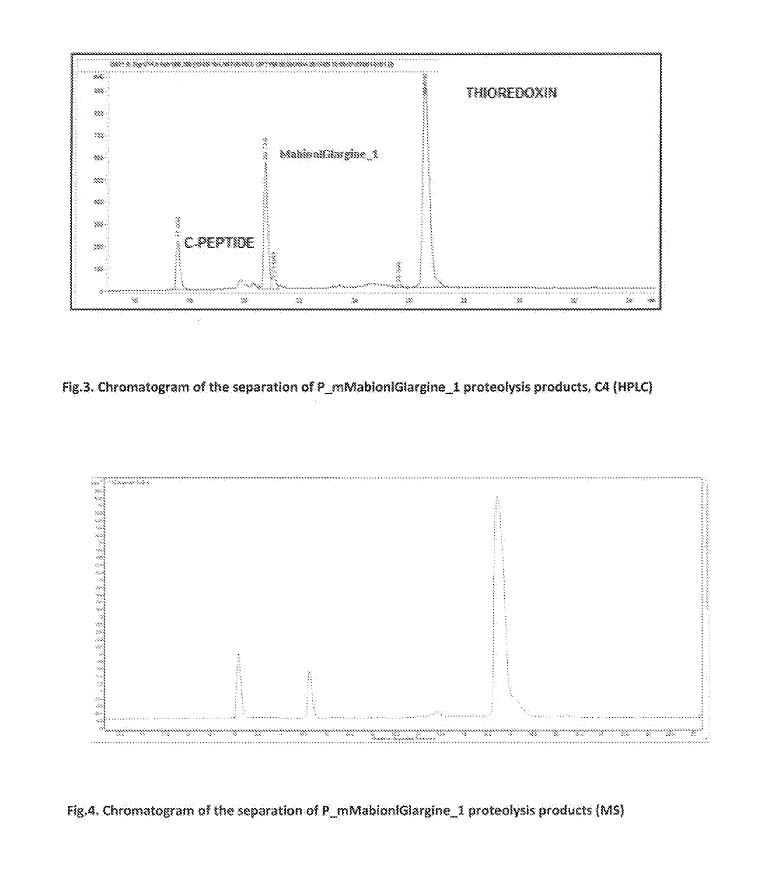

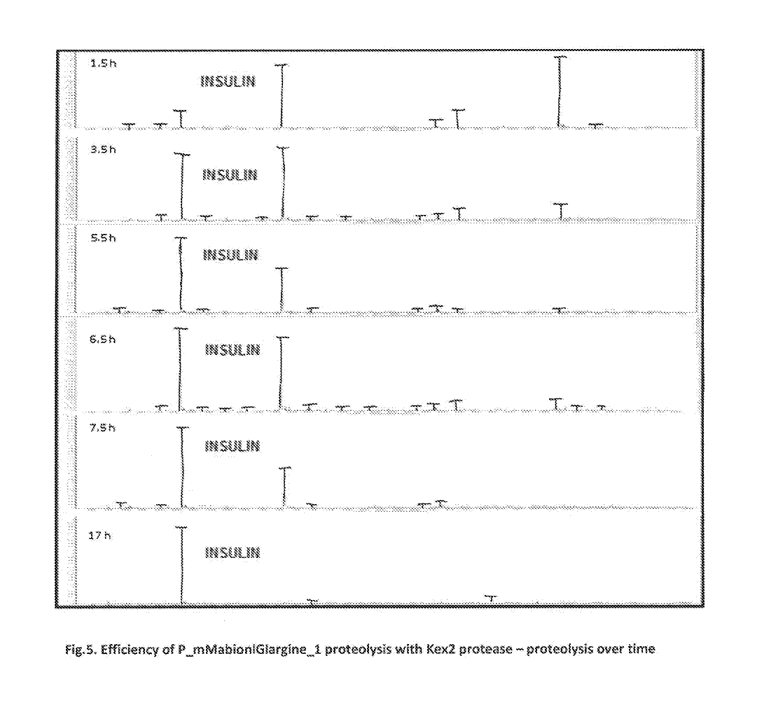

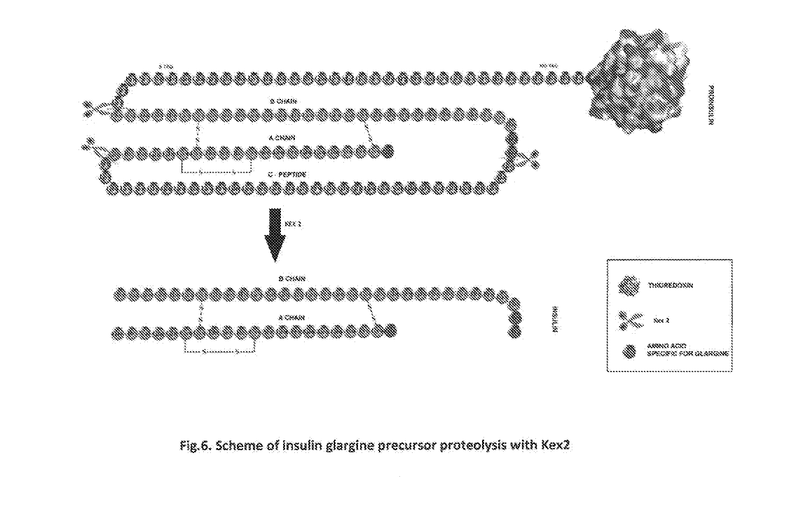

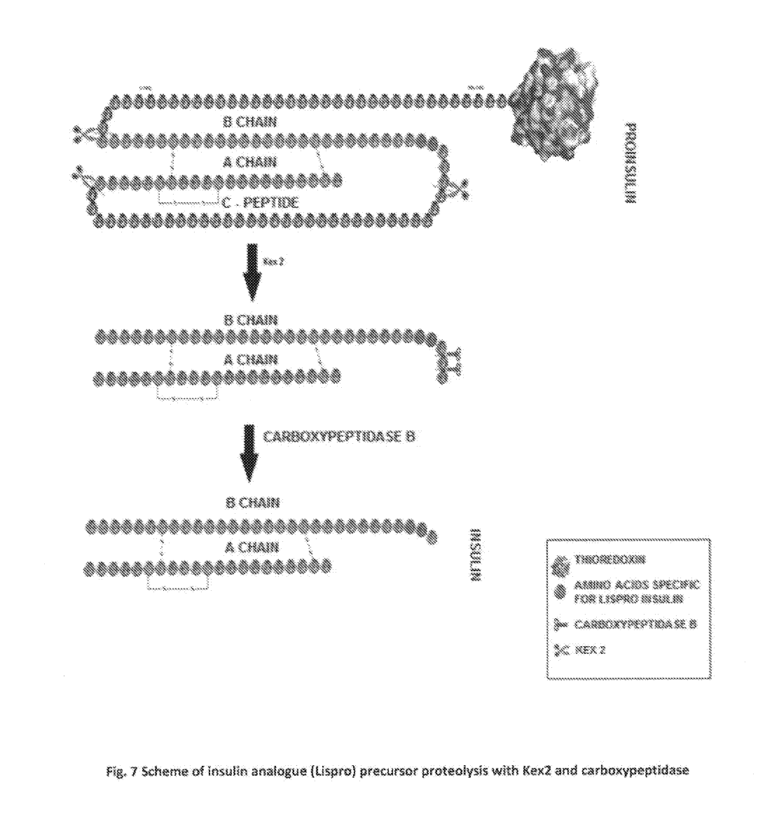

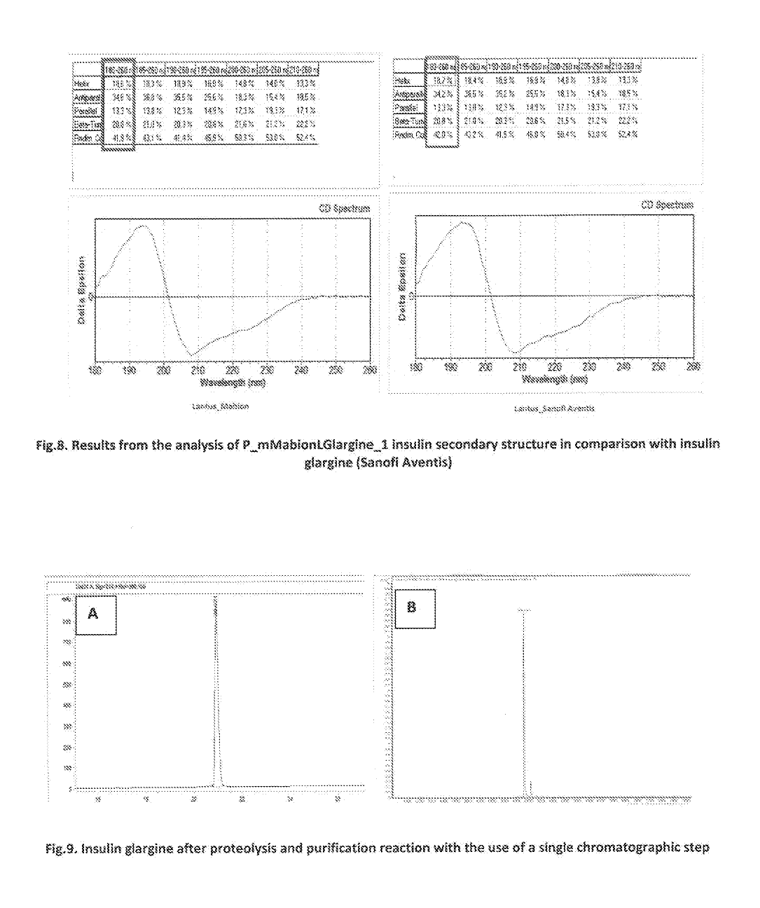

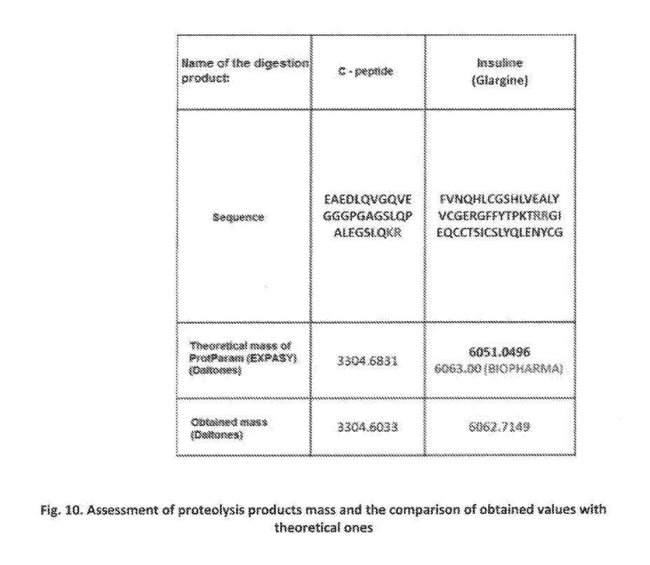

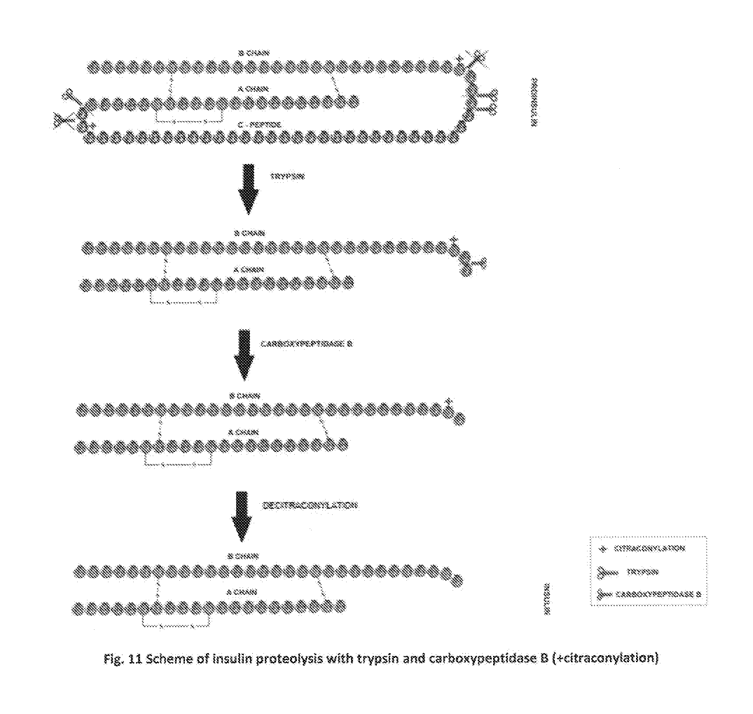

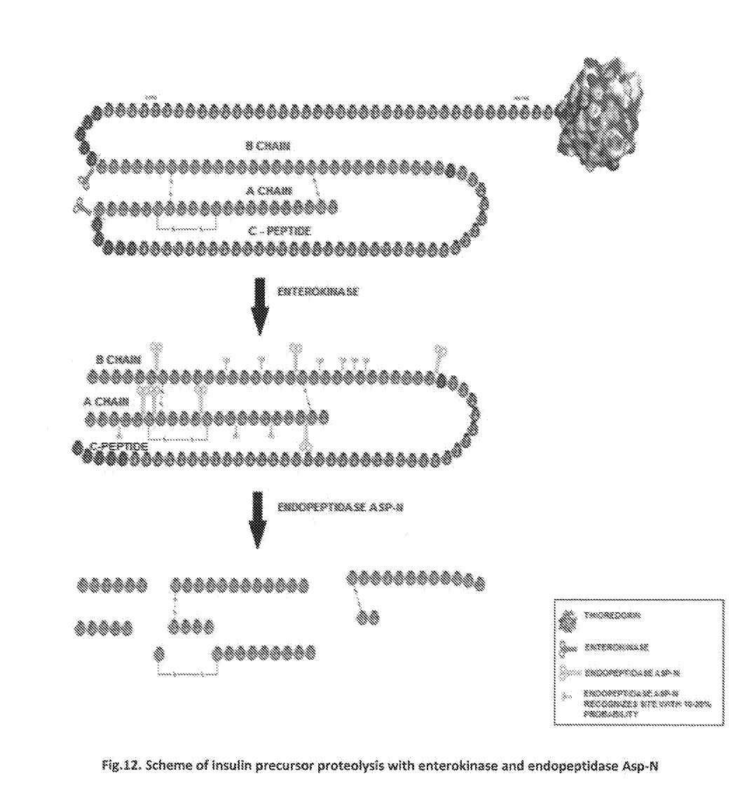

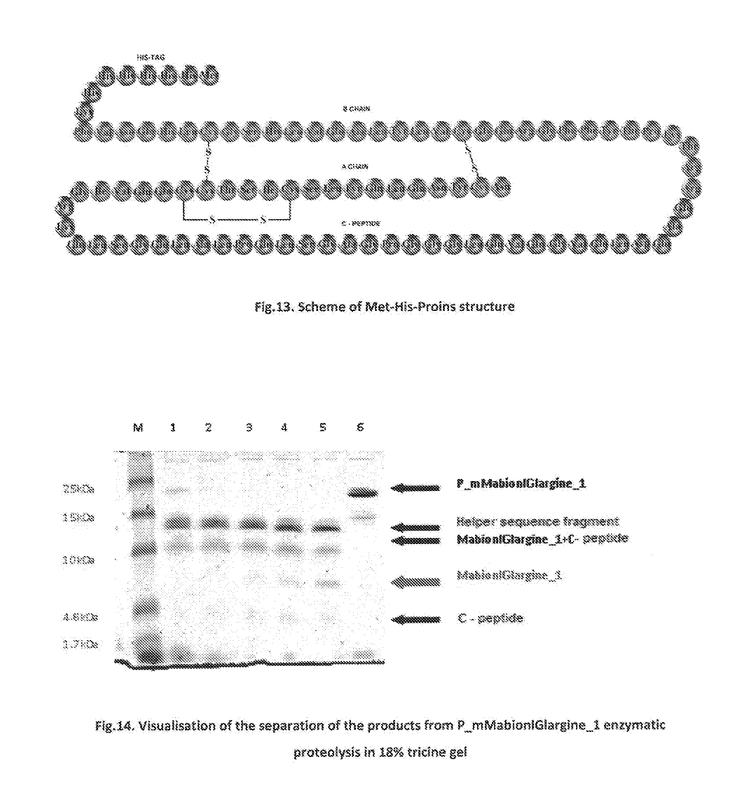

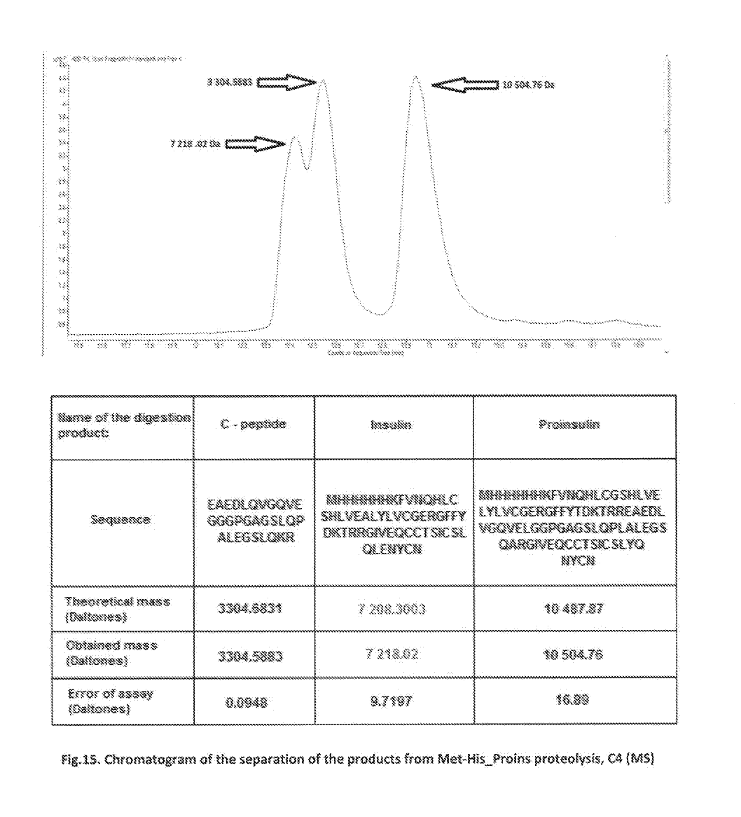

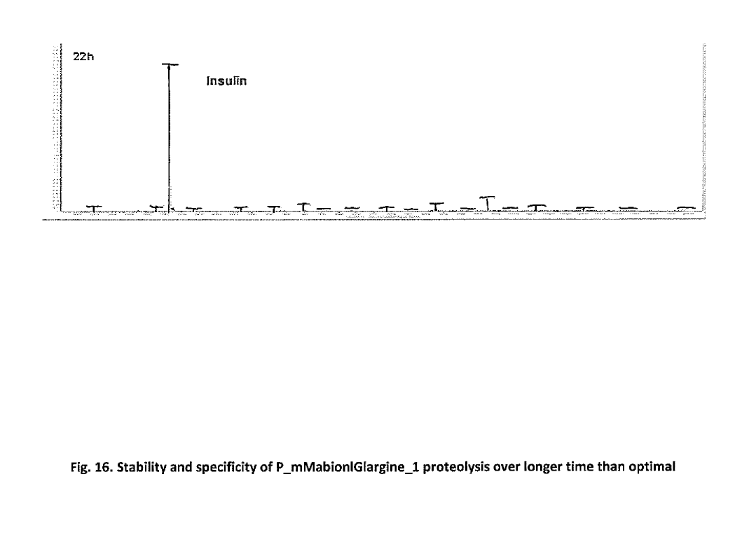

### DETAILED DESCRIPTION

Exemplary methods described herein offer even more efficient proteolysis than the one based on using Asp-N endoproteinase enterokinase. Asp-N endoproteinase (both the one from Flavobacterium meningosepticum as well as from Pseudomonasfragi) is an enzyme which hydrolyses peptide bonds preceding such amino acids as C=Cys, E=Glu, F=Phe, Y=Tyr. All of the mentioned amino acids are present in proinsulin or proinsulin analogue sequences. Thus, as a result of the Asp-N endoproteinase activity, many non-specific products occur.

As is generally known, protein molecules vary a lot in terms of both the structure and properties. The term of process universality is not a correct one in the context of biotechnological drugs production. The unique sequence of each protein imparts to it characteristic traits thanks to which it is possible to design the process of its production, starting from the fermentation step to the final purification step. Quite often it is possible to use some of the process steps to obtain protein with similar properties. However, it is not a case that using all the process steps could fully and without additional modifications be employed for obtaining a different protein product. Even a slight change in an amino acid sequence can positively or negatively affect physicochemical properties of a protein and, consequently, e.g. the efficiency of its purification. Thus, another advantage of using the described exemplary embodiments, also, the limitation of the scope of native sequence modifications. The design of the amino acid sequence of the proinsulin molecule (SEQ. ID. No. 4) consisted in the introduction of additional amino acids recognised by proteases: Asp-N and enterokinase. Consequently, C-peptide was elongated with six amino acids, out of which five were negatively charged amino acids, changing significantly the charge of the protein molecule. The key role of C-peptide is to bind insulin A chain with B chain and enabling the formation of disulphide bridges between the chains. C-peptide is a confirmed example of a proinsulin folding intramolecular “chaperone”. Amino acid sequences of proinsulin C-peptides differ depending on the species of origin. Nevertheless, C-peptide conservative fragments are important for maintaining the correct structure and solubility of the precursor, regardless of the species. These include, among others, N′-end acidic region and C′-end pentapeptide. A point mutation in any of N′-end amino acids (EAED) significantly affects the decrease in efficiency of in vitro protein folding compared with proinsulin in which C-peptide sequence was not modified. Thus, the change in the protein isoelectric point might be responsible for the decreased efficiency in protein folding. Hence, in case of P_MabionHI_1 proinsulin molecule we deal with interference in the protein pI value. The sequence analysis of proinsulin from different mammal species, including human, showed that the maximal number of acidic amino acids in C-peptide is five. The consequence of increasing the acidic amino acid content to ten is the decrease in the effectiveness of disulphide bridges formation. It is confirmed for example by the results obtained from the analysis of P_MabionHI_1 proinsulin proteolysis in which the prevalent amount of digestion products were free A and B chains in relation to actual insulin molecules. The described exemplary embodiment uses the native sequence of human proinsulin molecule or human proinsulin analogues. Lack of interference in the qualitative and quantitative amino acid content of C-peptide ensures obtaining the highest possible efficiency in disulphide bridges formation during fermentation.

In the prior art, it is known to use Kex2 serine protease, naturally present i.a. in yeast, to obtain insulin. Due to its cell localisation and characteristic digestion specificity, Kex2 protease is engaged in the proteolytic treatment of protein precursors in yeast cell, e.g. consisting in the conversion of a profactor and protoxin to active molecules. The processing of yeast protein precursors takes place in the Golgi apparatus in whose membrane Kex2 protease is localised. Apart from endogenic yeast proteins, protease efficiently converts recombinant proteins which are formed by expression of a gene introduced to a cell externally in a form of genetic construct—cDNA/expression vector. Such proteins include e.g. recombinant proalbumin. The high specificity of Kex2 comes from the digestion of a protein in whose sequence there are two specifically recognised amino acids (depending on the type of amino acid pair, Kex2 exhibits different digestion efficiency) which are recognised by it, and precisely after the second amino acid of a particular pair from the C′-end side it performs hydrolysis of the peptide bond. Yeasts are very often used in the production of recombinant proteins. The process in many cases consists in the protein secretion to the cell medium. The mechanism of secretion has several steps and requires suitable preparation of the target recombinant protein molecule. One of the standard modifications is using a leader sequence (α factor, MFα1 gene product). The a factor is commonly used as an additive (functioning as a signal peptide) for proteins produced in yeasts and then directed to the medium. Proinsulin in a native form is not readily expressed in Saccharomyces cerevisae. Yeast cells do not have traits characteristic for mammalian P cells, that is, large amount of rough endoplasmic reticulum, high concentration of zinc ions and storage vesicles which serve to accumulate insulin in a half-crystallised form. All of this is crucial for proper and efficient insulin secretion. Therefore, using the yeast system, the proinsulin molecule has to be initially subjected to required modifications, so that its efficient synthesis and production are possible. It is not necessary in case of using an expression system other than a yeast one, as in the description of an exemplary embodiment. Both insulin and its precursor molecule have hydrophobic character, and hence, they very readily aggregate in neutral pH. Glycosylation of the molecule obtained in this expression system is thus important for the formation of conformationally proper proinsulin and for the proper biosynthesis. It is a kind of a post-translational modification naturally occurring in yeast cells. However, what is crucial is the site of the mentioned glycosylation in case of proinsulin molecules. Therefore, in case of using yeast systems, the addition of the leader sequence—a factor to proinsulin sequence is an important change introduced to recombinant proteins. It is subjected to glycosylation in three sites, giving for the entire precursor the hydrophilic character and hence protecting it from aggregation. Further, adding a factor to the proinsulin molecule allows it to freely move between the borders of particular cell compartments engaged in protein processing. Nevertheless, this modification is not sufficient enough to achieve maximal efficiency in the production of the protein with proper conformation. It is necessary to additionally change the sequence of proinsulin molecule itself. Very often the C-peptide sequence is substantially shortened (even to three amino acids, e.g. AAK). Also the deletion of threonine from B chain (THrB30), which may be glycosylated in yeasts, is of importance. In this particular case, glycosylation is not desired, because insulin molecule in the final formulation does not have any modified amino acids. All these insulin molecules having sugar residues attached to ThrB30 would be hence a by-product of fermentation, exposing the entire process to losses. Finally, proinsulin is obtained which is expressed at a satisfying level with the high efficiency of sulphide bridges formation maintained. The fragment enabling secretion binds to the proper proinsulin molecule with a pair of amino acids KR/RR. This allows to use the activity of Kex2 protease naturally present in the Golgi apparatus. Kex2 specifically recognises the site for its proteolytic activity between the signal peptide and B chain (KR/RR). Subsequently, proinsulin efficiently secreted out of the yeast cell. To sum up, in the above fermentation process_Thr30B⁻ insulin precursor is obtained (that is, the precursor of desThr30B insulin). This precursor is then subjected to proteolysis with trypsin whose aim is to remove the sequence binding A and B chain. Additionally, a two-step stage is used to reconstitute missing threonine (Thr30B⁻). Initially, a reaction of desThr30B insulin with threonine ester takes place, and then the ester is removed by means of chemical hydrolysis. The production of proinsulin in such a form is the object of inventions in patent applications no. U.S. Pat. No. 4,916,212, WO 95/02059 and WO 90/10075. All mentioned modifications, necessary to be used for efficient production of proinsulin and insulin in yeasts, are not required in the case of using other expression system, e.g. bacterial ones.

Kex2 protease can be also used for complete conversion of human proinsulin or human proinsulin analogue to insulin or its analogue in yeast cell conditions, as shown in the description of patent application no. US 2011/0111460 A1. The C-peptide sequence was designed so that its length is optimal to achieve maximal folding efficiency. Above all, however, the sequence harboured two sites (amino acids pairs of KR or RR) specifically recognised by Kex2 protease, allowing to remove C-peptide. The essence of this technology is the secretion of human insulin molecules or its analogues to the culture medium. To achieve that, the second naturally present in yeasts protease is used, Kex1. It specifically removes two amino acids from the C′-end of B chain remaining after digestion with Kex2. Proposed solution allows to obtain insulin and insulin analogues in the final form which does not require the proteolysis step in in vitro conditions. The main downside, resulting from using such a technological approach, is low process efficiency, which is directly connected with the specificity of yeasts cells. This host, mostly due to pH conditions which prevail in its cell, is not able to maintain the monomeric form of insulin molecules. Also, using the yeast host for recombinant protein production the already mentioned problem connected with the occurrence of posttranslational modification, namely glycosylation. Mannosyltransferases present in Saccharomyces cerevisae attach oligosaccharide fragments to hydroxyl serine and threonine residues of both endogenic and recombinant proteins. Thus, a portion of proinsulin molecules secreted to the cell medium is glycosylated. Solutions proposed and described in U.S. Pat. No. 4,916,212, WO 95/02059, WO 90/10075 and US 2011/0111460 A1 do not refer to the matter of the contamination of proinsulin or insulin molecules pool with glycosylated derivatives of these molecules. In case of insulins and their analogues, glycosylated derivatives form significant contamination related to the product, and thus, cause major difficulties in the process. First of all, there are losses due to the necessity of removing glycosylated forms, secondly, purification itself is complicated and expensive. Therefore, the yeast cell is not an ideal host for the insulin or insulin analogue production process both in terms of the number of additional steps of the process, consisting in the abolishment of artificially introduced protein modifications, as well as in terms of the change in the profile of additional contamination with derivatives of the main product. In total, it has adverse effects on the efficiency of the process and its duration and cost.

Despite the availability of known methods for the production of insulins and their analogues, there is still a need for providing a method which would allow to obtain these proteins and would lack the above mentioned downsides which characterise known methods. Particularly, there is a need for providing a method more specific in relation to the existing ones as well as a more efficient one.

An exemplary method provides for preparation of a recombinant protein from a precursor, characterised in that a protease is used which hydrolyses one or more peptide bonds in this protein, wherein the protease disrupts the peptide bond from the C-end side of basic amino acid when this amino acid is the second one after other basic or neutral amino acid, and such an order enables specific recognition of both amino acids by the protease.

In an exemplary method, the protein obtained after the treatment with protease is subjected to the treatment with the second protease which is exopeptidase hydrolysing one or more peptide bonds of basic amino acid at the protein C-end.

In an exemplary method, the protein recombined from the precursor is insulin.

In an exemplary method, the protein recombined from the precursor is insulin analogue.

In an exemplary method, the pair of amino acids is a neutral and basic amino acid.

In an exemplary method, the pair of amino acids recognised by the protease is two basic amino acids, such as lysine-arginine or arginine-arginine or lysine-lysine.

In an exemplary method, the protease is Kex2 used in in vitro conditions.

In an exemplary method, the exoprotease is Kex1 or carboxypeptidase B in the in vitro use.

In an exemplary method, the organism in which the recombinant protein precursor is expressed is other than the yeast organism.

In an exemplary method, the proteolysis reaction is conducted in TrisHCl buffer conditions.

In an exemplary method, TrisHCl buffer concentration is 10-500 mM.

In another exemplary method, TrisHCl buffer concentration is 30-100 mM.

In an exemplary method, the proteolysis reaction is conducted in TrisHCl buffer conditions at pH of 5.5-8.5.

In another exemplary method, pH is 7.0-8.5.

In an exemplary method, the proteolysis reaction is conducted in the presence of NaCl at a concentration of 0-500 mM.

In another exemplary method, NaCl concentration is 0-50 mM.

In an exemplary method, the proteolysis reaction is conducted in the presence of CaCl₂ at a concentration of 0.1-20 mM.

In another exemplary method, CaCl₂ concentration is 1-10 mM.

In an exemplary method, the proteolysis reaction is conducted in the presence of glycerol at a concentration of 0.5-20%.

In another exemplary method, glycerol concentration is 5-15%.

In an exemplary method, the proteolysis reaction is conducted at the quantitative ratio of recombinant protein precursor to protease equal to 1:5-1:1000.

In another exemplary method, the quantitative ratio of recombinant protein precursor to protease is 1:10-1:400.

The advantage of the exemplary methods is the use in in vitro conditions an enzyme with specificity higher than used so far (e.g. trypsin or endopeptidase Asp-N), characterised in that the high specificity of precursor protein digestion is obtained. The use of the exemplary methods significantly limits the amount of digestion impurities related to the product by-products which influence the decrease in the process efficiency, they extend it and increase its costs. A new enzyme used in the process of obtaining insulin and insulin analogues from suitable precursors in in vitro conditions is Kex2 protease. Apart from high specificity, the protease is also characterised by proteolytic stability (lack of activity beyond the described specificity), even in case of extended incubation with a substrate. All of this has a tremendous effect on the reduction of by-product amounts and thus on the lack of the necessity to use additional protein purification steps. Special interest should be paid to the method for obtaining insulin analogue-insulin glargine in which case the proteolytic treatment of the precursor using Kex2 protease is a single-step one. In case of insulin and insulin analogues other than insulin glargine, it was found that a pair of proteases Kex2+carboxypeptidase B or Kex2+Kex1 provides more specific method for preparation of insulin and its analogues than any other protease/pair of proteases used thus far. Moreover, the advantage of obtaining insulin and insulin analogues by using Kex2 is the lack of necessity to introduce modifications in proinsulin molecule on DNA molecule level, because it naturally harbours amino acid sequences recognised by this enzyme. What is extraordinary and unusual in case of other proteases is the fact of enzymatic activity strictly connected with cutting only in case of compatibility of the two successive amino acids, arginine (P1), from the side where hydrolysis takes place, and another arginine or lysine (P2). Arginine in P1 is necessary, but in P2 changes are possible. However, the presence of different amino acid than lysine in P2 decreases the likelihood of peptide bond hydrolysis from a two-fold for arginine in P2 to 10⁻⁶ times for tryptophan. In the proinsulin molecule there are two amino acid pairs naturally occurring RR (arginine-arginine) and KR (lysine-arginine) which allow to cut off the C-peptide and form actual insulin molecule. The high specificity of Kex2 protease is a guarantee of high efficiency in digestion for insulin and its all analogues, wherein for insulin and most of its analogues other than insulin glargine it is necessary to use the second enzyme, exopeptidase cutting off amino acids from the C-end of protein molecule (specifically K, R or H). These are in some embodiments Kex1 and carboxypeptidase B.

An additional advantage which comes from using the exemplary embodiments is the possibility to add any amino acid sequence (e.g. a signal peptide) before B chain of the precursor, with the possibility to readily and specifically remove it during the precursor proteolysis step. Adding two amino acids, e.g. KR to which Kex2 has the highest affinity, before the first amino acid of insulin B chain N′-end and after the helper sequence fragment (SEQ. ID. No. 7) is the only interference, of little importance for the spatial structure, in proinsulin or insulin analogue sequence. Thanks to it, from insulin or insulin analogue precursor the helper sequence fragment and C-peptide are removed in one proteolytic step. In case of the process for preparation of insulin glargine analogue, it is the only proteolytic step leading to the production of the fully functional molecule. None of the said modifications has adverse effect on the charge of the actual proinsulin or proinsulin analogue molecule, or on the ability to form disulphide bridges. Introducing the modification to the insulin precursor molecule does not generate any additional steps of molecule processing (such as, e.g. citraconylation, chemical hydrolysis or additional purification step).

Efficient and specific proteolysis allows to significantly decrease the costs of insulin and insulin analogues purification, mostly through the reduction in the number of purification steps. In general, purification is a step which generates the highest costs in the whole process of protein drug production. They are incomparably higher than those which are generated in the fermentation step. In case of insulin and insulin analogues obtained according to the standard methods, apart from the need to remove the C-peptide from actual proteolysis product, additionally there is also the need to remove its derivatives generated as a result of undesired trypsin activity or formed during citraconylation/decitraconylation. In case of proteolysis with the use of Kex2 or Kex2 and carboxypeptidase B, the problem of contaminants related to product derivatives is not relevant, and the purification of insulin and its analogues is very often possible even in one chromatographic step, hence the possibility to reduce costs which favourably affects the economy of the entire process. The used protease is stable and does not change its activity over time, which is a kind of novelty in comparison to commonly used enzymes. Even with the time of digestion reaction extended, no by-products of Kex2 protease non-specificity are observed. It provides protection for molecules of insulin and its analogues during the proteolysis step. The reduction of this cost will be also influenced by the lack of glycosylated derivatives of insulin molecules connected with the use of bacterial host for protein production. Removing glycosylated derivatives of insulin or its analogues would generate the need for introducing additional chromatographic step and conducting a difficult and exposed to losses step of final product purification.

### EXAMPLES

Exemplary embodiments utilizing the principals described herein are further illustrated by the following examples, which are set forth to illustrate the presently disclosed subject matter and are not to be construed as limiting.

### Example 1

Preparation of Human Insulin from Human Proinsulin—Proteolysis with Trypsin and Carboxypeptidase B

The amino acid sequence of human insulin precursor is presented as SEQ. ID. No. 1. The protein amino acid sequence corresponds to the reference sequence, it was not modified. The human insulin precursor, IL_1, was obtained in Eschericha coli cells. In proteolysis reaction two proteases were used. Trypsin is an endopeptidase engaged in the step of proinsulin proteolytic treatment, consisting in the removal of C-peptide from the precursor. Endopeptidase specifically recognises single basic amino acids in the sequence: arginine (R) and lysine (K) and hydrolyses the peptide bond yR-|-x or yK-|-x, wherein x is any amino acid. The cutting efficiency is determined by the presence of R or K (amino acid y) amino acid at the N′-end, as it can significantly influence the occurrence of hydrolysis of the peptide bond from C′-end R or K. The proinsulin human molecule comprises in its sequence both arginine and lysine. Preferred proteolysis sites, unfortunately, are not the only ones recognised by trypsin. In the proinsulin molecule sequence, precisely in its B chain, there is lysine [LysB29] which also constitutes a site recognised by trypsin. Unfortunately, such digestion is undesirable. The second enzyme used in this reaction is carboxypeptidase B, an exopeptidase removing specifically basic amino acids from the protein C′-end R, K and H. The proteolysis reaction of 10 μg of Pre_IL_1 precursor was conducted in conditions of 50 mM Tris-HCl buffer, 1 mM CaCl₂, pH 7.6. Trypsin was used at the weight ratio of 1:400, while carboxypeptidase B in the weight ratio of 1:2000. Whereas, in said weight ratios a particular protease was used in amount of 400 or 2000 times less, respectively, than proinsulin. The reaction was conducted at the temperature of 37° C. for 1 hour. The analysis of the reaction results performed with reversed-phase high-pressure chromatography (C4, Vydac chromatographic bed was used, Phenomenex) and mass spectrometry confirmed the presence of the actual insulin molecule. Moreover, the presence of other molecules was observed, so called derivatives of the actual insulin product which significantly influenced reaction's final efficiency. Their presence is undesirable, because they constitute contamination related to the product. The decrease in proteolysis reaction efficiency directly results from the specificity of endoprotease used, trypsin. Due to its activity, apart from insulin, other reaction products were: insulin-des-ThrB30 and insulin-Arg65. Because of the presence of additional molecules (particularly in case of insulin-des-ThrB30) another step, consisting in the purification of actual insulin molecule, requires the use of additional chromatographic step. This affects both the costs of the process as well as its duration and efficiency.

### Example 2

Preparation of Human Insulin from Human Proinsulin—Proteolysis with Trypsin and Carboxypeptidase B (Additional Steps of the Process—Citraconylation/Decitraconylation)

The amino acid sequence of human insulin precursor is presented as SEQ. ID. No. 1. The protein amino acid sequence corresponds to the reference sequence, it was not modified. The human insulin precursor, Pre_IL_1, was obtained in Eschericha coli cells. In proteolysis reaction two proteases were used. Trypsin is an endopeptidase engaged in the step of proinsulin proteolytic treatment, consisting in the removal of C-peptide from the precursor. Endopeptidase specifically recognises single basic amino acids in the sequence: arginine (R) and lysine (K) and hydrolyses the peptide bond yR-|-x or yK-|-x, wherein x is any amino acid. The cutting efficiency is determined by the presence of R or K (amino acid y) amino acid at the N′-end, as it can significantly influence the occurrence of hydrolysis of the peptide bond from C′-end R or K. The proinsulin human molecule comprises in its sequence both arginine and lysine. Preferred proteolysis sites, unfortunately, are not the only ones recognised by trypsin. In the proinsulin molecule sequence, precisely in its B chain, there is lysine [LysB29] which also constitutes a site recognised by trypsin. Unfortunately, such digestion is undesirable. The second enzyme used in this reaction is carboxypeptidase B, an exopeptidase removing specifically basic amino acids from the protein C′-end R, K and H. In order to increase the specificity and thus desired activity of trypsin, an additional step of the process was introduced, consisting of two reactions. They resulted in a characteristic modification of the insulin precursor molecule (citraconylation/decitraconylation). Citraconylation was conducted prior to proteolysis reaction. IL_1 precursor was treated with citraconic or maleic acid anhydride, due to which, by acetylation reaction, the lysine residue (LysB29) was blocked. The change of the amino acid positive charge (LysB29) into negative prevents from hydrolysis of K⁻-|-T peptide bond in B chain. Conditions of the performed citraconylation: 10 μg of IL_1 precursor, 30 μg of citraconic or maleic acid anhydride, 50 mM Tris-HCl buffer, pH 8.4-8.5, 25° C., 2 h. The proteolysis reaction of 10 μg of IL_1 precursor was conducted in conditions of 50 mM Tris-HCl buffer, 1 mM CaCl₂, pH 7.6. Trypsin was used at the weight ratio of 1:400, while carboxypeptidase B in the weight ratio of 1:2000. Whereas, in said weight ratios a particular protease was used in amount of 400 or 2000 times less, respectively, than Pre_IL_1. The reaction was conducted at the temperature of 37° C. for 1 hour. Decitraconylation, conducted after precursor proteolysis, consisted in unblocking LysB29 residue and restoring the initial positive charge. The change in LysB29 charge was possible through the change in protein mixture pH to extremely acidic value equal to 2.5 and incubation of proinsulin in these conditions for 3 h. The analysis of the reaction results performed with reversed-phase high-pressure chromatography (C4, Vydac chromatographic bed was used, Phenomenex) and mass spectrometry confirmed the presence of the actual insulin molecule. Yet, the presence of other molecules was still observed, so called derivatives of the proper insulin product or contaminants related to the product, which significantly influenced final efficiency of the reaction. Although it was managed to reduce the amount of derivative, insulin-des-ThrB30, by 5-10% in relation to the amounts obtained without LysB29 modification, it is still present, similarly to insulin-Arg65. Because of the presence of additional molecules (particularly in case of insulin-des-ThrB30) another step, consisting in the purification of actual insulin molecule, requires the use of additional chromatographic step. This affects both the costs of the process as well as its duration and efficiency.

### Example 3

Preparation of Human Insulin from Human Proinsulin—Proteolysis with Enterokinase and Endoproteinase Asn

The amino acid sequence of human insulin precursor is presented as SEQ. ID. No. 1. The sequence was modified by the introduction of additional amino acids: DDDDK before the first amino acid of insulin A chain and D after the last amino acid of B chain (Thr30B), as presented below—SEQ. ID. No. 2. Additional DDDDK amino acids in the proinsulin molecule, similarly to those DDDDK in the helper sequence fragment, are specifically recognised by enterokinase, while D by endoproteinase Asp-N. The helper fragment, composed of peptides facilitating the protein purification, desirably affecting the protein solubility, enabling the protein purification with affinity chromatography and digestion with enterokinase, is presented as SEQ. ID. No. 3 and was added before the actual human insulin precursor molecule. The human insulin precursor together with the helper sequence fragment is SEQ. ID. No. 4.

The human insulin precursor, P_MabionHI_1, was obtained in Eschericha coli cells. During the digestion reaction no. I (with enterokinase) the helper sequence fragment or other fragment attached to the insulin molecule or other protein with DDDDK is efficiently removed. The C-peptide removal is possible thanks to conducting the digestion reaction no. II (with endoproteinase Asp-N). Enterokinase recognises DDDDK amino acid sequence with high specificity. The peptide bond hydrolysed during the reaction is DDDDK-|-x, wherein x is any amino acid. Endoproteinase Asp-N hydrolyses the peptide bond of the N′-end of aspartic acid: x-|-D, wherein x is any amino acid. Endoproteinase Asp-N shows also proteolytic activity towards N′-end peptide bond of such amino acids as: cysteine (C), glutamic acid (E), phenylalanine (F), tyrosine (Y), wherein it shows the highest activity towards aspartic acid (D). The proteolysis reaction no. I of 10 μg of P_MabionHI_1 precursor was conducted in the conditions of: 50 mM Tris-HCl buffer, pH 8.0. Enterokinase was used in the amount of 0.05 μg. The reaction was conducted at the temperature of 37° C. for 16 hours. The reaction no. II with endopeptidase Asp-N was conducted in the conditions of: 50 mM Tris-HCl buffer, 2.5 mM ZnSO₄, pH 8.0, at the temperature of 25° C. for two hours. After the reaction no. I, the appropriate amount of endopeptidase Asp-N—0.15 μg was added to the protein mixture. The analysis of the results from both reactions performed with reversed-phase high-pressure chromatography (C4, Vydac chromatographic bed was used, Phenomenex) and mass spectrometry confirmed the presence of the actual insulin molecule. In the reaction mixture there is also a large pool of peptides formed as a result of non-specific endopeptidase Asp-N activity. The change in the C-peptide length caused the lack of or incorrect formation of S—S bonds, resulting in free A and B chains and/or fragments of free A and B chains observed in the mixture after the digestion reaction. It is difficult to assess the efficiency of the above proteolysis reactions (no. I and no. II) due to the large amount of non-specific products. The quantity and variety of contaminants related to the product make it more difficult to design as well as conduct an effective step of insulin purification. The need for introducing additional chromatographic steps extends the duration of the entire purification process and significantly increases its costs.

### Example 4

Preparation of Human Met-HisTag-Proinsulin from Modified Human Proinsulin—Proteolysis with Kex2

The amino acid sequence of human Met-HisTag-proinsulin precursor is presented as SEQ. ID. No. 5. Amino acids in the insulin precursor sequence in the setting: KR, similarly to RR pair of amino acids, are specifically recognised by Kex2 protease. Thanks to that, it is possible to remove the C-peptide in a single reaction. The helper fragment, in this case Met and HisTag peptide, are not relevant in the analysis, they probably enabled gene expression and precursor purification. The human insulin precursor, Met-HisTag-proinsulin, commercially available (R&D Systems), was prepared according to the manufacturer's instructions. Obtaining human Met-HisTag-insulin from the precursor is possible thanks to the use of highly specific proteolysis reaction. During the digestion reaction, the C-peptide precursor is efficiently removed. The C-peptide removal is possible thanks to dipeptides, respectively, two arginine (RR) at the C-peptide N′-end and lysine and arginine (KR) at the C-peptide C′-end, which naturally occur in the human insulin precursor molecule. Kex2 protease recognises RR and KR dipeptides with high specificity. The peptide bond hydrolysed during the reaction is, respectively, RR-|-x or KR-|-x, wherein x is any amino acid apart from valine (V). The proteolysis reaction of 3 μg of Met-HisTag-proinsulin precursor was conducted in the conditions of 50 mM Tris-HCl buffer, 1 mM CaCl₂, pH 7.0. Kex2 protease was used in the amount of 0.3 μl [0.3 μg/0.3 l], wherein 1 μg of the enzyme is 0.04 U. The reaction was conducted at the temperature of 37° C. for 16 hours. The analysis of the reaction results performed with reversed-phase high-pressure chromatography (C4, Vydac chromatographic bed was used, Phenomenex) and mass spectrometry confirmed the experiment hypothesis. Kex2 protease separated the human insulin molecule (M-HisTag-B—RR and A chain) from C-peptide in the efficient way and with high specificity. No by-products were identified which would constitute contaminants related to the product.

### Example 5

Preparation of Human Insulin from Modified Human Proinsulin—Proteolysis with Kex2 and Carboxypeptidase B

The amino acid sequence of human insulin precursor is presented as SEQ. ID. No. 1. The sequence was modified by the introduction of two additional amino acids KR (lysine and arginine) before the first amino acid of the insulin B chain, as shown below—SEQ. ID. No. 6. Additional amino acids (KR), similarly to RR pair of amino acids, are specifically recognised by Kex2 protease. Thanks to that, it is possible to remove the helper sequence fragment and C-peptide in a single reaction. The helper fragment, composed of peptides facilitating the protein purification, desirably affecting the protein solubility, enabling the protein purification with affinity chromatography, is presented as SEQ. ID. No. 3 and was added before the actual human insulin precursor molecule. The human insulin precursor together with the helper sequence fragment is SEQ. ID. No. 7.

The human insulin precursor, P_MabionHI_1, was obtained in Eschericha coli cells. During the digestion reaction, the helper sequence fragment or other fragment attached to the insulin molecule or other protein by the pair of amino acids: lysine and arginine (KR) or two arginine (RR) as well as the C-peptide precursor are efficiently removed. The C-peptide removal is possible thanks to dipeptides, respectively, two arginine (RR) at the C-peptide N′-end and lysine and arginine (KR) at the C-peptide C′-end, which natively occur in the human insulin precursor molecule. Kex2 protease recognises RR and KR dipeptides with high specificity. The peptide bond hydrolysed during the reaction is, respectively, RR-|-x or KR-|-x, wherein x is any amino acid apart from valine (V). The proteolysis reaction no. I of 10 μg of P_mMabionIL_precursor was conducted in the conditions of 25 mM Tris-HCl buffer, 1 mM CaCl₂, pH 7.7. Kex2 protease was used in the amount of 1 μl [1 μg/1 μl], wherein 1 μg of the enzyme is 0.04 U. The reaction was conducted at the temperature of 37° C. for 16 hours. The analysis of the results from reaction no. I performed with reversed-phase high-pressure chromatography (C4, Vydac chromatographic bed was used, Phenomenex) and mass spectrometry confirmed the experiment hypothesis. Kex2 protease separated the human insulin molecule (B—RR and A chain) from C-peptide and helper sequence fragment. The removal of two arginine from the B chain C′-end was possible thanks to the use of exopeptidase—carboxypeptidase B or Kex1, specifically removing basic amino acids: arginine (R), lysine (K) and histidine (H) from the protein molecule C′-end. The reaction mixture after the proteolysis reaction no. I was the initial protein solution for the reaction no. II. To the reaction environment, NaCl at the final concentration of 0.1 M and carboxypeptidase B at the weight ratio of 1:2000 (assuming enzyme activity within the range of 90.0%-99.9%) were added, and the proteolysis reaction was conducted at the temperature of 37° C. for one hour. As a result of the reaction no. II, the actual human insulin molecule (SEQ. ID. No. 8) was obtained, what was confirmed by the sequence and mass analysis. No by-products were identified which would constitute contaminants related to the product.

### Example 6

Preparation of Human Insulin Analogues from Modified Analogues of Human Proinsulin—Proteolysis with Kex2 and Carboxypeptidase B (in Case of Lispro Analogue)

The amino acid sequence of human insulin precursor is presented as SEQ. ID. No. 1. The sequence was modified by changing ProB28B with LysB29 and introducing two additional amino acids KR (lysine and arginine) before the first amino acid of the insulin B chain, as shown below—SEQ. ID. No. 9. Additional amino acids (KR), similarly to the pair of amino acids, arginine (RR), are specifically recognised by Kex2 protease. Thanks to that it is possible to remove the helper sequence fragment and C-peptide in a single reaction. The helper fragment, composed of peptides facilitating the protein purification, desirably affecting the protein solubility, enabling the protein purification with affinity chromatography, is presented as SEQ. ID. No. 3 and was added before the actual human insulin precursor molecule. The human insulin precursor together with the helper sequence fragment is SEQ. ID. No. 10.

The human insulin precursor, P_mMabionILispro_1, was obtained in Eschericha coli cells. Preparation of the human insulin from the insulin precursor is possible thanks to the use of highly specific proteolysis reaction. During the digestion reaction, the helper sequence fragment or other fragment attached to the insulin molecule or other protein by KR or RR as well as the C-peptide precursor are efficiently removed. The C-peptide removal is possible thanks to dipeptides, respectively, RR at the C-peptide N′-end and KR at the C-peptide C′-end, which natively occur in the human insulin precursor molecule. Kex2 protease recognises RR and KR dipeptides with high specificity. The peptide bond hydrolysed during the reaction is, respectively, RR-|-x or KR-|-x, wherein x is any amino acid apart from valine (V). The proteolysis reaction no. I of 10 μg of P_mMabionILispro_1 precursor was conducted in the conditions of 25 mM Tris-HCl buffer, 1 mM CaCl₂, pH 7.7. Kex2 protease was used in the amount of 1 μl [1 μg/1 μl], wherein 1 μg of the enzyme is 0.04 U. The reaction was conducted at the temperature of 37° C. for 16 hours. The analysis of the results from reaction no. I performed with reversed-phase high-pressure chromatography (C4, Vydac chromatographic bed was used, Phenomenex) and mass spectrometry confirmed the experiment hypothesis. Kex2 protease separated the human insulin molecule (B—RR and A chain) from C-peptide and helper sequence fragment. The removal of two arginine from the B chain C′-end was possible thanks to the use of exopeptidase—carboxypeptidase B or Kex1, specifically removing basic amino acids: arginine (R), lysine (K) and histidine (H) from the protein molecule C′-end. The reaction mixture after the proteolysis reaction no. I was the initial protein solution for the reaction no. II. To the reaction environment, NaCl at the final concentration of 0.1 M and carboxypeptidase B at the weight ratio of 1:2000 (assuming enzyme activity within the range of 90.0%-99.9%) were added, and the proteolysis reaction was conducted at the temperature of 37° C. for one hour. As a result of the reaction no. II, the actual human insulin molecule (SEQ. ID. No. 11) was obtained, what was confirmed by the sequence and mass analysis. No by-products were identified which would constitute contaminants related to the product.

### Example 7

Preparation of Human Insulin Analogue with Prolonged Activity (Insulin Glargine)

The amino acid sequence of human insulin precursor is presented as SEQ. ID. No. 1. The sequence was modified by introducing two additional amino acids KR (lysine and arginine) before the first amino acid of the insulin B chain and changing A21N amino acid into A21G, as shown below—SEQ. ID. No. 12. Additional amino acids (KR) are specifically recognised by Kex2 protease, thus it is possible to remove the helper sequence fragment in a single reaction. The helper fragment, composed of peptides facilitating the protein purification, desirably affecting the protein solubility, enabling the protein purification with affinity chromatography, is presented as SEQ. ID. No. 3 and was added before the actual human insulin precursor molecule. The human insulin precursor together with the helper sequence fragment is SEQ. ID. No. 13.

The human insulin precursor, P_mMabionlGlargine_1, was obtained in Eschericha coli cells. Preparation of the human insulin analogue from the insulin analogue precursor is possible thanks to the use of highly specific proteolysis reaction. During it, the helper sequence fragment or other fragment attached to the insulin molecule or other protein by KR or RR as well as the C-peptide precursor are efficiently removed. The C-peptide removal is possible thanks to dipeptides, respectively, RR at the C-peptide N′-end and KR at the C-peptide C′-end, which natively occur in the human insulin precursor molecule. Kex2 protease recognises RR and KR dipeptides with high specificity. The peptide bond hydrolysed during the reaction is, respectively, RR-|-x or KR-|-x, wherein x is any amino acid apart from valine (V). The proteolysis reaction no. I of 10 μg of P_mMabionlGlargine_1 precursor was conducted in the conditions of 25 mM Tris-HCl buffer, 1 mM CaCl₂, pH 7.4. Kex2 protease was used in the amount of 1 μl [1 μg/1 μl], wherein 1 μg of the enzyme is 0.04 U. The reaction was conducted at the temperature of 37° C. for 16 hours. The analysis of the results from reaction no. I performed with reversed-phase high-pressure chromatography (C4, Vydac chromatographic bed was used, Phenomenex) and mass spectrometry confirmed the experiment hypothesis. Kex2 protease separated the human insulin molecule (B—RR and A chain) from C-peptide and helper sequence fragment. No by-products were identified which would constitute contaminants related to the product. In case of this insulin analogue, the reaction no. I is the only proteolytic step necessary to convert the insulin analogue precursor (SEQ. ID. No. 14) to the actual insulin analogue molecule.

The following references are incorporated by reference herein:
1. IDF Diabetes Atlas 6th edition, 2013, International Diabetes Federation
2. “Deaths: Final Data for 2010” Statistics Reports, vol 61 no 4, Aug. 5, 2013, CDC, National Center for Health Statistics
3. WHO, Diabetes Fact sheet no 312, October 2013.
4. Dolan-Heitinger J., Recombinant DNA and Biosynthetic Human Insulin, A Source Book, Eli Lilly and Co.: Indianapolis, 1982; Barfoed H. C., Insulin Production Technology, Chem. Eng. Prog., 1987, 83 (10), 49-54.
5. Marglin A., Insulin and solid chase synthesis, 1964-1970, Peptide Science 2007, 90(3), 200-2002
6. Barfoed H. C., Insulin Production Technology, Chem. Eng. Prog., 1987, 83 (10), 49-54.
7. Ladish M. R., Kohlmann K. L., Recombinant Human Insulin, Biotechnol. Prog., 1992, 8 (6), 469-478.
8. Johnson I. S., Human Insulin from Recombinant DNA Technology, 1983, Science, 219, 632-637.
9. Williams D. C., Van Frank R. M., Muth W. L., Burnett J. P., Cytoplasmic inclusion Dobies In Escherichia coli producing biosynthetic human insulin proteins, 1982, Science, 215 (5), 687-689.
10. Chance R. E., Kroeff E. P., Hoffman J. A., W: Insulins, Growth Hormone and Recombinant DNA Technology, Raven Press, New York, 1981, pp. 71-85.
11. Ladish M. R., Kohlmann K. L., Recombinant Human Insulin, Biotechnol. Prog., 1992, 8 (6), 469-478.
12. Chance R., Glazer N., Wishner K., Insulin Lispro (Humalog). W: Walsh G., Murphy B. (red) Biopharmaceuticals, an industrial perspective, Kluwer, Dordrecht 1999, pp 149-172.
13. Son Y J, Kim C K, Kim Y B, Kweon D H, Park Y C, Seo J H, Effects of Citraconylation on Enzymatic Modification of Human Proinsulin Using Trypsin and Carboxypeptidase B, 2009, Biotechnol. Prog., 25(4), 1064-1070.
14. Nielsen R. G., Sittampalam G. S., Rickard E. C., Capillary zone electrophoresis of insulin and growth-hormone, 1989, Anal Biochem., 177, 20-26.
15. Habermann P., Zocher F., Method for producing insulin analogs having a dibascic chain B terminus, US Patent Application Publication no US 2009/0192073 A1.
16. Shahravan S H, Qu X, Chan I, Shin J A, 2008, Protein Expr Purif., 59(2), 314-319.
17. Tarentino A L, Quinnes G, Grimwood B G, Hauer C R, Plummer T H, Molecular cloning and sequence analysis of Flavastacin: An O-Glycosylated Procaryotic Zinc Metalloendopeptidase, 1995, Archives of Biochemistry and Biophisics, 319(1), 281-285.
18. Substrate Specificity of a Proteolytic Enzyme Isolated from a Mutant of Pseudomonas fragi, Drapeau G R, 1980, The Journal of Biological Chemistry, 255(3), 839-840), (Ingrosso D, Fowler A V, Bleibaum J, Clarke S, 1989, Specificity of endoproteinase Asp-N(Pseudomonas fragi): cleavage at glutamyl residues in two proteins, Biochemical and Biophysical Research Communications, 162(3), 1528-1534.
19. Qiao Z, Min C, Hua Q, Weiss M A, Feng Y, 2003, In Vitro Refolding of Human Proinsulin, The Journal of Biological Chemistry, 278(20), 17800-17809.
20. Chen L, Yang X, Tang J, 2002, Acidic Residues on the N-terminus of Proinsulin C-Peptide Are Important for the Folding of Insulin Precursor, J. Biochem, 131, 855-859.
21. Steiner D F, 2004, The Proinsulin C-peptide—A Multirole Model, Experimental Diab., 5, 7-14.
22. Fuller R S, Brake A, Thorner J, 1988, Yeast prohormone processing enzyme (Yeast prohormone processing nzyme (KEX2 gene product) is a Ca²⁺-dependent serine protease, Proc. Natl. Acad. Sci., 86, 1434-1438.
23. Wilcox C A, Fuller R S, 1991, Posttranslational Processing of the Prohormone-cleaving Kex2 Protease in the Saccharomyces cerevisae Secretory Pathway, The Journal of Cell Biology, 115(2), 297-307.
24. Ledgerwood E C, George P M, Peach R J, Brennan S O, 1995, Endoproteolytic processing of recombinant proalbumin variants by the yeast Kex2 protease, Biochem. J., 308, 321-325.
25. Kjeldsen T, Balschmidt P, Diers I, Hach M, Kaarsholm N C, Ludvigsen S, 2001, Expression of Insulin In Yeats: The importance of Molecular Adaptation for Secretion and Conversion, Biotechnology and Engineering Reviews, 18.
26. Kjeldsen T, Frost Pettersson A, Hach M, 1999, Secretory expression and characterization of insulin in Pichia pastoris.
27. Gentzsch M, Tanner W, 1997, Protein-O-glycosylation in yeast: protein-specific mannosyltransferases, Glycobiology, 7(4), 481-486.
28. Kjeldsen T, 2000, Yeast secretory expression of insulin precursors, Appl. Microbiol. Biotechnol., 54, 277-286.
29. Bevan A, Brenner C, Fuller A S, 1998, Quantitative assessment of enzyme specificity in vivo: P2 recognition by Kex2 protease defined in a genetic system, Biochemistry, 95, 10384-10389.

1-22. (canceled)

23. A method for preparation of insulin or insulin analogue from a recombinant protein precursor, comprising
a) using a protease Kex2 in in vitro conditions,
wherein the protease hydrolyzes one or more peptide bonds in the precursor,
wherein the protease disrupts a peptide bond from a C-end side of a basic amino acid,
wherein in the precursor the amino acid having the disrupted peptide bond is in an order such that it is a second one after another basic or neutral amino acid,
wherein such order of the amino acids enables specific recognition of both amino acids by the protease, and
b) expressing in bacteria the resultant insulin or insulin analogue precursor.

24. The method according to claim 23, wherein in (b) the bacteria in which the insulin or insulin analogue precursor is expressed is Escherichia coli.

25. The method according to claim 23, and further comprising
prior to (b) subjecting the protein obtained in (a) to treatment with a second protease which is an exopeptidase,
wherein the exopeptidase hydrolyzes one or more peptide bonds of a basic amino acid at a protein C-end,
wherein the protein C-end is Kex1 or carboxypeptidase B in vitro.

26. The method according to claim 23, wherein in (a) the amino acids are both neutral and basic amino acids.

27. The method according to claim 24, wherein in (a) the amino acids are both neutral and basic amino acids.

28. The method according to claim 25, wherein in (a) the amino acids are both neutral and basic amino acids.

29. The method according to claim 23, wherein in (a) the protease recognizes the amino acids, wherein the amino acids consist of a pair of basic amino acids, such pair including lysine-arginine or arginine-arginine or lysine-lysine.

30. The method according to claim 24, wherein in (a) the protease recognizes the amino acids, wherein the amino acids consist of a pair of basic amino acids, such pair including lysine-arginine or arginine-arginine or lysine-lysine.

31. The method according to claim 25, wherein in (a) the protease recognizes the amino acids, wherein the amino acids consist of a pair of basic amino acids, such pair including lysine-arginine or arginine-arginine or lysine-lysine.

32. The method according to claim 23,
wherein (a) is conducted in TrisHCl buffer conditions.

33. The method according to claim 32, wherein the TrisHCl buffer concentralion is 30-100 mM.

34. The method according to claim 33, wherein (a) is conducted in TrisHCl buffer conditions at pH of 7.0-8.0.

35. The method according to claim 34, wherein (a) is carried out with a KCl or NaCl concentration in the range of 25-50 mM.

36. The method according to claim 35, wherein (a) is carried out with a CaCl₂ concentration in the range of 1-10 mM.

37. The method according to claim 36, wherein (a) is carried out with a glycerol concentration in the range of 0.5-15%.

38. The method according to claim 37, wherein in (a) a quantitative ratio of the recombinant protein precursor to the protease is in the range of 1:10-1:100.


SEQUENCE LISTING (14 sequences)

SEQ ID NO: 1 - protein, 86 aa, Artificial sequence
  human proinsulin (Pre_IL_l)
FVNQHLCGSHLVEALYLVCGERGFFYTPKTRREAEDLQVGQVELGGGPGAGSLQPLALEGSLQKRGIVEQCCTSICSLYQLENYCN

SEQ ID NO: 2 - protein, 92 aa, Artificial sequence
  MabionHI_1
FVNQHLCGSHLVEALYLVCGERGFFYTPKTDRREAEDLQVGQVELGGGPGAGSLQPLALEGSLQKRDDDDKGIVEQCCTSICSLYQLENYCN

SEQ ID NO: 3 - protein, 153 aa, Artificial Sequence
  helper sequence fragment (z pET32a(+))
MSDKIIHLTDDSFDTDVLKADGAILVDFWAEWCGPCKMIAPILDEIADEYQGKLTVAKLNIDQNPGTAPKYGIRGIPTLLLFKNGEVAATKVGALSKGQLKEFLDANLAGSGSGHMHHHHHHSSGLVPRGSGMKETAAAKFERQHMDSPDLGT

SEQ ID NO: 4 - protein, 251 aa, Artificial Sequence
  P_MaibonHI_1
MSDKIIHLTDDSFDTDVLKADGAILVDFWAEWCGPCKMIAPILDEIADEYQGKLTVAKLNIDQNPGTAPKYGIRGIPTLLLFKNGEVAATKVGALSKGQLKEFLDANLAGSGSGHMHHHHHHSSGLVPRGSGMKETAAAKFERQHMDSPDLGTDDDDDKFVNQHLCGSHLVEALYLVCGERGFFYTPKTDRREAEDLQVGQVELGGGPGAGSLQPLALEGSLQKRDDDDKGIVEQCCTSICSLYQLENYCN

SEQ ID NO: 5 - protein, 94 aa, Artificial Sequence
  Met-HisTag-proinsulin
MHHHHHHKFVNQHLCGSHLVEALYLVCGERGFFYTPKTRREAEDLQVGQVELGGGPGAGSLQPLALEGSLQKRGIVEQCCTSICSLYQLENYCN

SEQ ID NO: 6 - protein, 88 aa, Artificial sequence
  mMabioniL_1
KRFVNQHLCGSHLVEALYLVCGERGFFYTPKTRREAEDLQVGQVELGGGPGAGSLQPLALEGSLQKRGIVEQCCTSICSLYQLENYCN

SEQ ID NO: 7 - protein, 241 aa, Artificial Sequence
  P_mMabionIL_1
MSDKIIHLTDDSFDTDVLKADGAILVDFWAEWCGPCKMIAPILDEIADEYQGKLTVAKLNIDQNPGTAPKYGIRGIPTLLLFKNGEVAATKVGALSKGQLKEFLDANLAGSGSGHMHHHHHHSSGLVPRGSGMKETAAAKFERQHMDSPDLGTKRFVNQHLCGSHLVEALYLVCGERGFFYTPKTRREAEDLQVGQVELGGGPGAGSLQPLALEGSLQKRGIVEQCCTSICSLYQLENYCN

SEQ ID NO: 8 - protein, 51 aa, Artificial sequence
  MabionIL_l
FVNQHLCGSHLVEALYLVCGERGFFYTPKTGIVEQCCTSICSLYQLENYCN

SEQ ID NO: 9 - protein, 88 aa, Artificial Sequence
  mMabionILisPro_1
KRFVNQHLCGSHLVEALYLVCGERGFFYTKPTRREAEDLQVGQVELGGGPGAGSLQPLALEGSLQKRGIVEQCCTSICSLYQLENYCN

SEQ ID NO: 10 - protein, 241 aa, Artificial Sequence
  P_mMabionILisPro_1
MSDKIIHLTDDSFDTDVLKADGAILVDFWAEWCGPCKMIAPILDEIADEYQGKLTVAKLNIDQNPGTAPKYGIRGIPTLLLFKNGEVAATKVGALSKGQLKEFLDANLAGSGSGHMHHHHHHSSGLVPRGSGMKETAAAKFERQHMDSPDLGTKRFVNQHLCGSHLVEALYLVCGERGFFYTKPTRREAEDLQVGQVELGGGPGAGSLQPLALEGSLQKRGIVEQCCTSICSLYQLENYCN

SEQ ID NO: 11 - protein, 51 aa, Artificial Sequence
  mMabionILisPro_1
FVNQHLCGSHLVEALYLVCGERGFFYTKPTGIVEQCCTSICSLYQLENYCN

SEQ ID NO: 12 - protein, 88 aa, Artificial Sequence
  mMabionIGlargine_1
KRFVNQHLCGSHLVEALYLVCGERGFFYTPKTRREAEDLQVGQVELGGGPGAGSLQPLALEGSLQKRGIVEQCCTSICSLYQLENYCG

SEQ ID NO: 13 - protein, 241 aa, Artificial Sequence
  P_mMabionIGlargine_1
MSDKIIHLTDDSFDTDVLKADGAILVDFWAEWCGPCKMIAPILDEIADEYQGKLTVAKLNIDQNPGTAPKYGIRGIPTLLLFKNGEVAATKVGALSKGQLKEFLDANLAGSGSGHMHHHHHHSSGLVPRGSGMKETAAAKFERQHMDSPDLGTKRFVNQHLCGSHLVEALYLVCGERGFFYTPKTRREAEDLQVGQVELGGGPGAGSLQPLALEGSLQKRGIVEQCCTSICSLYQLENYCG

SEQ ID NO: 14 - protein, 53 aa, Artificial Sequence
  MabionIGlargine_1
FVNQHLCGSHLVEALYLVCGERGFFYTPKTRRGIVEQCCTSICSLYQLENYCG